(19) United States
(12) Patent Application Publication
(10) Pub. No.: US 2025/0143581 A1
(43) Pub. Date: May 8, 2025

(54) ULTRASONIC IMAGING SYSTEM AND SYNCHRONIZATION CONTROL METHOD

(71) Applicant: FUJIFILM Corporation, Tokyo (JP)

(72) Inventors: Hirozumi TAKESHIMA, Tokyo (JP); Tomohiko TANAKA, Tokyo (JP)

(73) Assignee: FUJIFILM Corporation, Tokyo (JP)

(21) Appl. No.: 18/935,620

(22) Filed: Nov. 3, 2024

(30) Foreign Application Priority Data
Nov. 7, 2023 (JP) 2023-189866

(51) Int. Cl. A61B 5/00

(52) U.S. Cl. CPC A61B 5/0095; A61B 5/7235

(57) ABSTRACT

An ultrasonic imaging system is provided. An insertion member to be inserted into a living body includes an optical absorption element that absorbs an optical pulse to generate a photoacoustic wave. The analyzer calculates synchronization deviation based on a photoacoustic wave signal sequence included in a reception signal sequence before phase addition. The synchronization controller changes the optical pulse period or the reception period based on the synchronization deviation. The synchronization deviation may be calculated based on a pseudo-reception signal sequence corresponding to the reception signal sequence.

### CROSS-REFERENCE TO RELATED APPLICATION

This application claims priority under 35 USC 119 from Japanese Patent Application No. 2023-189866, filed 7 Nov. 2023, the disclosure of which is incorporated by reference herein.

### BACKGROUND OF THE INVENTION

### 1. Field of the Invention

The present disclosure relates to an ultrasonic imaging system and a synchronization control method, and particularly, to a technique for displaying a position of an insertion object inserted into a living body.

### 2. Description of the Related Art

An ultrasonic imaging system is a system for performing treatment or examination on a living body which is a patient using an ultrasonic imaging apparatus. More specifically, in the ultrasonic imaging system, an image representing a position of an insertion member inserted into the living body is generated and displayed. The treatment or the like is performed on the living body with reference to such an image. From such a viewpoint, the ultrasonic imaging system is a system for supporting treatment or the like on a living body.

In the ultrasonic imaging system, an ultrasound diagnostic apparatus is usually used as the ultrasonic imaging apparatus. The insertion member to be inserted into the living body is, for example, a catheter to be inserted into a blood vessel. In general, a guide wire is inserted into a blood vessel before the insertion of the catheter. The guide wire is also the insertion member. A treatment with an ultrasonic imaging system that images an insertion member inserted into a blood vessel using an ultrasound probe that is in contact with a surface of a living body is also referred to as an extra-vascular ultrasound (EVUS).

An advanced ultrasonic imaging system that images an insertion member using a photoacoustic effect has been proposed. In such an ultrasonic imaging system, an optical absorption element is provided at a distal end of the insertion member. An optical pulse generated by an optical pulse generator is guided to the inside of the insertion object through an optical fiber, and the optical pulse is emitted to the optical absorption element. A photoacoustic wave is generated in the living body by the absorption of the optical pulse by the optical absorption element. The photoacoustic wave is received by an ultrasound probe that is in contact with a biological surface. An image (hereinafter, referred to as a photoacoustic image or a PA image) representing the position of the optical absorption element (that is, the sound source) is formed based on reception information obtained by the photoacoustic wave. For example, the photoacoustic image is composited with the ultrasound image (hereinafter, also referred to as a US image) generated by the ultrasonic transmission and reception. As a result, the generated composite image is displayed. Through an observation of the composite image, the position of the distal end of the insertion member can be clearly specified while observing the biological tissue.

In the above-described advanced ultrasonic imaging system, it is necessary to precisely synchronize an optical pulse period in the optical pulse generator with the reception period in the ultrasound probe. For example, even in a case in which the sound source is located below the ultrasound probe, the sound source is not imaged in a case in which the timing at which the photoacoustic wave from the sound source reaches the ultrasound probe is outside the reception period. In addition, in a case in which the synchronization is not established, the reception beamforming is not appropriately executed, and a quality of the image is deteriorated.

JP5819387B discloses the above-described advanced ultrasonic imaging system. In the system, a method of electrically synchronizing reception and the optical pulse is adopted, that is, it is necessary to provide a special dedicated circuit for synchronization. Therefore, it can be pointed out that the system configuration is complicated. JP2012-29715A discloses a photoacoustic diagnostic apparatus. The photoacoustic diagnostic apparatus does not image the position of the insertion object inserted into the living body.

### SUMMARY OF THE INVENTION

An object of the present disclosure is to correctly synchronize an optical pulse period and a reception period while avoiding or reducing complication of a system configuration in an ultrasonic imaging system that images a position of an insertion member using a photoacoustic effect.

An ultrasonic imaging system according to the present disclosure comprises a light source configured to generate an optical pulse; an insertion member that is inserted into a living body and that includes an optical absorption element which converts the optical pulse into a photoacoustic wave; a probe including a plurality of transducers that receive the photoacoustic wave; a receiver configured to apply phase addition (beamforming) to a reception signal sequence consisting of a plurality of reception signals that are output in parallel from the plurality of transducers; a generator configured to generate a photoacoustic image representing a position of the optical absorption element in the living body based on reception information output from the receiver; an analyzer configured to analyze the reception signal sequence or a pseudo-reception signal sequence corresponding to the reception signal sequence to calculate a synchronization deviation between an optical pulse period in the light source and a reception period in the probe; and a controller configured to change at least one of the optical pulse period or the reception period based on the synchronization deviation.

A synchronization control method according to the present disclosure comprises a step of, in a state in which an insertion member including an optical absorption element that converts an optical pulse from a light source into a photoacoustic wave is inserted into an acoustic propagation medium and a probe is in contact with the acoustic propagation medium, receiving the photoacoustic wave from the optical absorption element via a plurality of transducers in the probe; a step of analyzing a reception signal sequence consisting of a plurality of reception signals output in parallel from the plurality of transducers or a pseudo-reception signal sequence corresponding to the reception signal sequence to calculate a synchronization deviation between an optical pulse period in the light source and a reception period in the probe; and a step of changing at least one of the optical pulse period or the reception period based on the synchronization deviation.

According to the present disclosure, it is possible to correctly synchronize an optical pulse period and a reception period while avoiding or reducing complication of a system configuration in an ultrasonic imaging system that images a position of an insertion member using a photoacoustic effect.

### BRIEF DESCRIPTION OF THE DRAWINGS

FIG. 1 is a block diagram showing an ultrasonic imaging system according to a first embodiment.

FIG. 2 is a timing chart showing a first example of the synchronization control method.

FIG. 3 is a diagram showing an example of a reception signal before phase addition.

FIG. 4 is a diagram showing an example of a reception signal sequence before phase addition.

FIG. 5 is a diagram showing a spatial relationship between a sound source and a transducer array.

FIG. 6 is a diagram showing a relationship between a synchronization deviation, a real propagation time, and an apparent propagation time.

FIG. 7 is a diagram showing an example of a synchronization deviation calculation method.

FIG. 8 is a flowchart showing a first example of a synchronization control method.

FIG. 9 is a timing chart showing a second example of the synchronization control method.

FIG. 10 is a flowchart showing the second example of the synchronization control method.

FIG. 11 is a block diagram showing an ultrasonic imaging system according to a second embodiment.

FIG. 12 is a diagram showing a first modification example.

FIG. 13 is a diagram showing a second modification example.

FIG. 14 is a flowchart showing an operation of the ultrasonic imaging system.

### DESCRIPTION OF THE PREFERRED EMBODIMENTS

Hereinafter, the embodiment will be described with reference to the drawings.

### (1) Summary of Embodiment

An ultrasonic imaging system according to the embodiment includes a light source, an insertion member, a probe, a receiver, a generator, an analyzer, and a controller. The light source generates an optical pulse. The insertion member is inserted into a living body and has an optical absorption element that converts the optical pulse into a photoacoustic wave. The probe comprises a plurality of transducers that receive the photoacoustic wave. The receiver applies phase addition to a reception signal sequence consisting of a plurality of reception signals that are output in parallel from the plurality of transducers. The generator generates a photoacoustic image representing a position of the optical absorption element in the living body based on reception information output from the receiver. The analyzer analyzes the reception signal sequence or a pseudo-reception signal sequence corresponding to the reception signal sequence, and thus calculates a synchronization deviation between an optical pulse period in the light source and a reception period in the probe. The controller changes at least one of the optical pulse period or the reception period based on the synchronization deviation.

With the above configuration, it is possible to specify the synchronization deviation by analyzing the reception signal sequence or the pseudo-reception signal sequence corresponding to the reception signal sequence generated by the reception of the photoacoustic wave. In addition, it is possible to correctly synchronize the optical pulse period and the reception period based on the specified synchronization deviation while avoiding or reducing complication of a system configuration in an ultrasonic imaging system.

One or both of the analyzer and the controller may be disposed in an ultrasonic imaging apparatus, an information processing apparatus, or an optical pulse generation apparatus including a light source. In a case in which a pulse period is changed based on the synchronization deviation, the pulse period is matched with the reception period. In a case in which the reception period is changed based on the synchronization deviation, the reception period is matched to the pulse period.

The synchronization deviation may be measured in a state in which the insertion member is inserted into the living body, or the synchronization deviation may be measured in a state in which the insertion member is inserted into an acoustic propagation medium other than the living body. The insertion member is a member for treatment or a member for examination. The pseudo-reception signal sequence can be considered to be the reception signal sequence described above from the viewpoint of calculating the synchronization deviation. For example, the pseudo-reception signal may be generated by applying a calculation that is the inverse of phase addition (inverse beamforming) to the reception information output from the receiver.

In the embodiment, the analyzer detects a plurality of photoacoustic wave signals included in the reception signal sequence or the pseudo-reception signal sequence, and calculates the synchronization deviation based on a plurality of detection timings of the plurality of photoacoustic wave signals. With this configuration, the synchronization deviation is calculated based on the timing at which the photoacoustic wave reaches each transducer. The photoacoustic wave signal appears as a peak or a pulse having a large amplitude in each reception signal or each pseudo-reception signal. It is relatively easy to specify each of the photoacoustic wave signals.

In the embodiment, the analyzer calculates a plurality of apparent propagation times from the reception period start timing to a plurality of detection timings, and calculates the synchronization deviation based on the plurality of apparent propagation times. Specifically, the analyzer calculates the synchronization deviation by giving a plurality of apparent propagation times to a mathematical model including the synchronization deviation parameter as the unknown parameter. The apparent propagation time is a time including a delay time corresponding to the phase deviation.

The synchronization deviation may be calculated by a method other than the method using the mathematical model. For example, the synchronization deviation may be calculated based on a shape in which the photoacoustic wave signal sequence draws in the reception signal sequence or the pseudo-reception signal sequence.

In the embodiment, the analyzer analyzes the reception signal sequence extracted from the receiver. The reception signal sequence to be extracted is the reception signal sequence before phase addition. Alternatively, the analyzer analyzes the pseudo-reception signal. In that case, a restorer that generates a pseudo-reception signal sequence corresponding to the reception signal sequence is provided based on the reception information.

In the embodiment, in a case in which the reception signal sequence or the pseudo-reception signal sequence does not include the photoacoustic wave signal sequence, the controller changes on a trial basis at least one of the optical pulse period or the reception period until the photoacoustic wave signal sequence is included in the reception signal sequence or the pseudo-reception signal sequence. For example, a new pulse period or a new reception period may be determined on a trial basis by adding a certain time to the pulse period or the reception period or by subtracting a certain time from the pulse period or the reception period.

In the embodiment, the controller changes at least one of the optical pulse period or the reception period based on the synchronization deviation in a preparation step before a main step of performing the treatment or the examination on the living body. The preparation step is a synchronization establishment step. In the preparation step, an acoustic propagation medium other than the living body is used as necessary. The main step is a treatment step or an examination step. In the main step, control for the calculation of the synchronization deviation and the synchronization maintenance may be executed.

The synchronization control method according to the embodiment includes a reception step, a calculation step, and a change step. In the reception step, in a state in which the insertion member including the optical absorption element that converts the optical pulse from the light source into the photoacoustic wave is inserted into the acoustic propagation medium and the probe is in contact with the acoustic propagation medium, the plurality of transducers in the probe receive the photoacoustic wave from the optical absorption element. In the calculation step, the reception signal sequence consisting of the plurality of reception signals output in parallel from the plurality of transducers or the pseudo-reception signal sequence corresponding to the reception signal sequence is analyzed, and thus the synchronization deviation between the optical pulse period in the light source and the reception period in the probe is calculated. In the change step, at least one of the optical pulse period or the reception period is changed based on the synchronization deviation.

### (2) Details of Embodiment

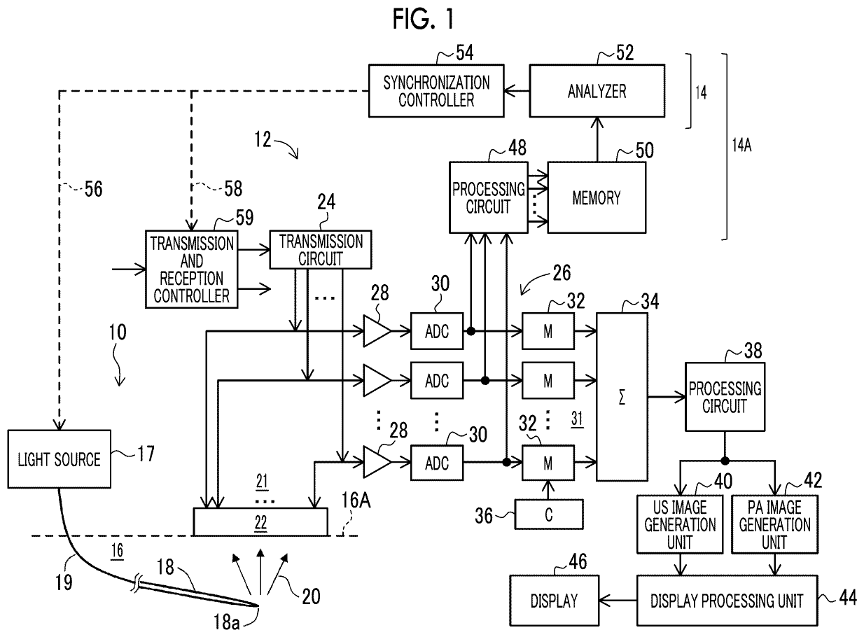

FIG. 1 shows an ultrasonic imaging system according to a first embodiment. The ultrasonic imaging system is a medical system installed in a medical institution such as a hospital, and specifically, is a system used in a case of sending a distal end of an insertion member to a diseased part while observing a photoacoustic image (PA image) representing a position of the distal end of the insertion member. The ultrasonic imaging system may be used for other applications.

The ultrasonic imaging system includes an optical pulse generation apparatus 10, an ultrasound diagnostic apparatus 12, an information processing apparatus 14, and an insertion member 18. The optical pulse generation apparatus 10 includes a light source 17. The light source 17 is a laser that generates a pulse sequence of laser light. A proximal end of the optical fiber 19 is connected to the light source 17. A distal end of the optical fiber 19 is located in a distal end part of the insertion member 18.

The insertion member 18 is, for example, a catheter inserted into a blood vessel in the living body 16. Other examples of the insertion member 18 include a guide wire, a puncture needle, or the like. An optical absorption element 18a made of an optical absorption material is provided in the distal end part of the insertion member 18. The optical absorption element 18a absorbs the optical pulse by irradiating the optical pulse onto the optical absorption element 18a. At that time, the photoacoustic wave 20 is generated by the photoacoustic effect. The photoacoustic wave 20 propagates through the living body 16 as a pulsed wave.

Next, the ultrasound diagnostic apparatus 12 as the ultrasonic imaging apparatus will be described. The ultrasound probe 21 is in contact with a surface 16A of the living body 16. The ultrasound probe 21 is held by an examiner or a robot. A transducer array 22 composed of a plurality of transducers is provided in the ultrasound probe 21.

In a case of forming a normal tomographic image, the ultrasound wave is transmitted into the living body 16 by the transducer array 22, and the reflected wave from the inside of the living body 16 is received by the transducer array 22. More specifically, a transmission beam and a reception beam are formed and are electronically scanned.

In a case of forming the photoacoustic image, the transducer array 22 performs only the reception operation without performing the transmission operation. That is, the photoacoustic wave generated in the living body 16 is received by the transducer array 22. More specifically, a plurality of reception periods are set on a time axis in accordance with the reception period. Each reception period is a period for receiving or detecting the photoacoustic wave, in other words, corresponds to the reception beam forming period.

It should be noted that a plurality of reception beams may be simultaneously and parallelly formed in each reception period. The reception signal sequence obtained from the entire transducer array 22 may be used as the reception information for the synchronization deviation analysis, and a part of the reception signal sequence may be used as the reception information for the reception beam formation. In general, the transmission and reception process for forming the tomographic image and the reception process for forming the photoacoustic image are alternately executed.

The transmission circuit 24 is a transmission beam former. That is, the transmission circuit 24 is an electronic circuit that outputs a plurality of transmission signals in parallel to the plurality of transducers during transmission.

The reception circuit 26 corresponds to a receiver or a reception unit. Specifically, the reception circuit 26 is an electronic circuit that processes a plurality of reception signals output in parallel from the plurality of transducers during reception. The reception circuit 26 includes a plurality of amplifiers 28 that amplify a plurality of reception signals, an ADC 30 that converts the amplified plurality of reception signals (analog signals) into a plurality of digital signals, and a phase addition unit (beamforming unit) 31 that applies phase addition to the converted plurality of reception signals. The phase addition is processing of generating reception beam data from the plurality of reception signals.

More specifically, the phase addition unit 31 includes a plurality of memories 32 that temporally store the converted plurality of reception signals, an adder 34 that adds the plurality of reception signals read out from the plurality of memories, and a controller 36 that controls writing of the plurality of reception signals to the plurality of memories 32 and that controls reading of the plurality of reception signals from the plurality of memories 32. The phases of the plurality of reception signals are aligned by controlling the readout timing of the plurality of reception signals from the plurality of memories 32. In the case of the phase addition, so-called reception dynamic focusing is performed, and so-called parallel reception is performed as necessary.

The reception circuit 26 outputs the reception beam data generated by the phase addition. A plurality of pieces of reception beam data arranged in an electronic scanning direction are generated by one electronic scanning of a reception beam. The reception frame data corresponding to the beam scanning plane is configured by the plurality of pieces of reception beam data. Each reception beam data is composed of a plurality of pieces of echo data arranged in a depth direction. It should be noted that, in the reception circuit 26, the reception beam data may be generated by software processing.

The processing circuit 38 is an electronic circuit that processes each reception beam data. The processing circuit 38 includes an envelope detection circuit, a filter circuit, a logarithmic conversion circuit, and the like. In a case of generating the ultrasound image (US image), each reception beam data output from the processing circuit 38 is sent to the US image generation unit 40. In a case of generating the photoacoustic image (PA image), each reception beam data output from the processing circuit 38 is sent to the PA image generation unit 42.

The US image generation unit 40 is a module that has a digital scan converter (DSC) and generates display frame data from the reception frame data. Specifically, the US image generation unit 40 generates a tomographic image (B-mode tomographic image) representing a tissue structure as the US image. The DSC has a coordinate transformation function, a pixel interpolation function, and the like.

The PA image generation unit 42 is a module that has the DSC and the sound source specifying unit and generates the display frame data from the reception frame data. The sound source specifying unit specifies a position of the sound source in the living body by detecting or extracting a sound source signal included in the display frame data generated by the DSC. The PA image includes a marker representing a position of the sound source in the beam scanning plane. The marker is, for example, a point having high brightness or a predetermined color.

The display processing unit 44 generates a composite image by superimposing the PA image on the US image (tomographic image). The composite image is displayed on the display 46. The display 46 is configured by an organic EL display device, a liquid crystal display device, or the like.

The US image generation unit 40, the PA image generation unit 42, and the display processing unit 44 are each configured by a processor. The CPU that controls the operation of the ultrasound diagnostic apparatus 12 may be allowed to function as the US image generation unit 40, the PA image generation unit 42, and the display processing unit 44.

The transmission and reception controller 59 controls the operations of the transmission circuit 24 and the reception circuit 26. The transmission and reception controller 59 determines a reception period and a time length of a reception period. The CPU may be allowed to function as the transmission and reception controller 59.

In the ultrasound diagnostic apparatus 12 according to the first embodiment, the reception signal sequence before phase addition is extracted from the reception circuit 26. The reception signal sequence is composed of the plurality of reception signals output from the plurality of ADCs 30. In the shown configuration example, the extracted plurality of reception signals are temporally stored in the memory 50 via the processing circuit 48. Each reception signal may be output from the processing circuit 48 to the information processing apparatus 14. Each reception signal may be directly output from the reception circuit 26 to the information processing apparatus 14.

The processing circuit 48 is an electronic circuit that applies necessary signal processing to each reception signal. The necessary signal processing may include envelope detection, noise removal processing, and the like. A processing circuit 48 and a memory 50 may be provided in the information processing apparatus 14 (see reference numeral 14A).

The information processing apparatus 14 is configured by, for example, a computer. The information processing apparatus 14 includes an analyzer 52 and a synchronization controller 54. The processor configures the above-described components. The CPU in the information processing apparatus 14 may be allowed to function as the analyzer 52 and the synchronization controller 54. The analyzer 52 and the synchronization controller 54 may be provided in the optical pulse generation apparatus 10 or the ultrasound diagnostic apparatus 12.

The analyzer 52 detects the photoacoustic wave signal sequence included in the reception signal sequence before phase addition, and calculates a synchronization deviation (synchronization deviation amount) between the optical pulse period and the reception period based on the photoacoustic wave signal sequence. The synchronization deviation can also be referred to as a phase deviation. The calculation method of the synchronization deviation will be described in detail below.

The synchronization controller 54 has a function of changing or correcting at least one of the optical pulse period or the reception period based on the calculated synchronization deviation, and thereby synchronizing the optical pulse period and the reception period (synchronization control function). In addition, the synchronization controller 54 has a function of changing the optical pulse period or the reception period on a trial basis (trial change function). In a case in which the photoacoustic wave cannot be observed due to the fact that the photoacoustic wave reaches the transducer array 22 during a period other than the reception period, the synchronization controller 54 changes on a trial basis at least one of the optical pulse period or the reception period.

In a case in which the optical pulse period is changed within the optical pulse period and the reception period, the control signal 56 is output to the optical pulse generation apparatus 10 from the synchronization controller 54. In a case in which the reception period is changed within the optical pulse period and the reception period, the control signal 58 is output to the transmission and reception controller 59 from the synchronization controller 54. Here, the reception period is a reception period for generating the PA image.

As will be described later, in the preparation step, the synchronization between the optical pulse period and the reception period is established. In the main step (treatment step or examination step) following the preparation step, the optical pulse period and the reception period are maintained. However, in the main step following the preparation step, the control for maintaining the synchronization establishment state may be continuously executed.

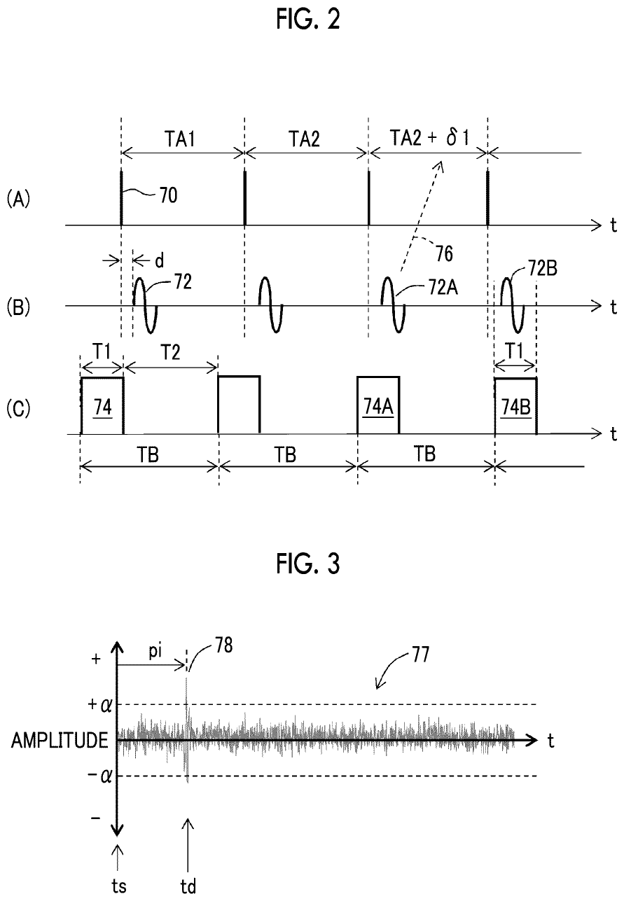

FIG. 2 shows a first example of the synchronization control as a timing chart. (A) shows an optical pulse sequence generated by the light source. The optical pulse sequence is composed of a plurality of optical pulses 70 arranged on a time axis. A width of the optical pulse is, for example, 100 ns, and the optical pulse period is, for example, 1 ms.

(B) shows a plurality of photoacoustic wave signal sequences 72 arranged on the time axis. In FIG. 2, each of the photoacoustic wave signal sequences 72 is schematically represented, that is, represented as a single pulse wave. A photoacoustic wave generated by one optical pulse is received by a plurality of transducers, so that a plurality of photoacoustic wave signals are generated in parallel. The photoacoustic wave signal sequence 72 is composed of a plurality of photoacoustic wave signals. d indicates a propagation time of the photoacoustic wave.

(C) shows a plurality of reception periods 74 arranged on the time axis. Each reception period 74 is a period in which the photoacoustic wave can be received. Each reception period 74 is set in the ultrasound probe, in other words, is set for the reception circuit. For example, the reception opening is set for the entire transducer array, and the reception signal sequence corresponding to the reception opening is analyzed.

In the first example shown in FIG. 2, the optical pulse period is changed within the optical pulse period and the reception period based on the calculated synchronization deviation. TA1, TA2, and TA2+δ1 each indicate an optical pulse period. TB indicates a reception period. The reception period TB is fixed. The time length of each reception period 74 is T1. By the way, T2 is a time length of a blank period.

The above-described synchronization controller changes on a trial basis the optical pulse period in a case in which the photoacoustic wave signal sequence 72 cannot be observed in the reception period 74. Specifically, the next optical pulse period TA2 is set by adding or subtracting a certain period to or from the optical pulse period TA1.

In the example shown in FIG. 2, the photoacoustic wave signal sequence 72A is observed in the reception period 74A. The analysis of the photoacoustic wave signal sequence 72A calculates the synchronization deviation 81 (see reference numeral 76), and the synchronization deviation 81 is added to the current optical pulse period TA2 to set a new optical pulse period TA2+δ1. As a result, synchronization is established between the optical pulse period and the reception period. Specifically, the photoacoustic wave signal sequence 72B is correctly observed in the reception period 74B. Thereafter, as necessary, the calculation of the synchronization deviation and the control based on the synchronization deviation are continuously executed to maintain the synchronization establishment state.

FIG. 3 shows an example of the reception signal before phase addition. The reception signal 77 includes a photoacoustic wave signal 78 generated due to the reception of the photoacoustic wave. The photoacoustic wave signal 78 has a peak-like or pulse-like form having a large amplitude. For example, the photoacoustic wave signal 78 can be detected or extracted by the threshold value processing. In that case, a portion exceeding the threshold value a is specified as the photoacoustic wave signal 78. The threshold value a may be set according to the magnitude of the noise included in the reception signal. For example, in a case in which the standard deviation of the reception signal is represented by o, the threshold value a may be set in accordance with α=6σ. Prior to the threshold value processing, filter processing, envelope detection, or the like may be applied to the reception signal.

By detecting the photoacoustic wave signal 78, the timing (detection timing) td at which the photoacoustic wave is detected is specified for each reception signal. The period pi from the reception period start timing ts to the detection timing td includes a time corresponding to the propagation time of the photoacoustic wave and the synchronization deviation. From such a viewpoint, in the following, the period pi will be referred to as an apparent propagation time.

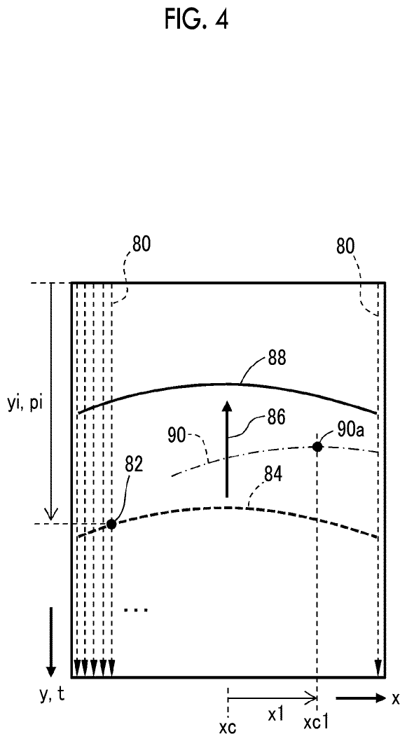

FIG. 4 shows an example of the reception signal sequence before phase addition. The reception signal sequence is composed of a plurality of reception signals 80 corresponding to the plurality of transducers constituting the transducer array. In FIG. 4, the x direction is a transducer array direction, and the y direction is a depth direction. The y direction corresponds to a time axis.

The reception signal sequence includes a photoacoustic wave signal sequence 84. In the shown example, the x coordinate (xc) of the center of the transducer array matches the x coordinate of the sound source, and the x coordinate of the apex of the photoacoustic wave signal sequence 84 matches the x coordinate (xc) of the center of the transducer array. The photoacoustic wave signal sequence 84 is composed of a plurality of photoacoustic wave signals. Focusing on a specific photoacoustic wave signal 82 received by the i-th transducer, the apparent propagation time pi is specified based on the detection timing. The specific photoacoustic wave signal 82 is generated at a point at a depth yi.

The photoacoustic wave signal sequence 84 has a parabolic form. The form of the photoacoustic wave signal sequence 84 is constant regardless of the magnitude of the synchronization deviation. A position at which the photoacoustic wave signal sequence 84 is generated varies depending on the spatial relationship between the transducer array and the sound source. For example, in a case in which a position of the sound source is shifted in the x direction from the center position xc of the transducer array, the photoacoustic wave signal sequence 90 is generated. An x coordinate (xc1) of the apex 90a corresponds to the x coordinate of the sound source. Even in that case, the form of the photoacoustic wave signal sequence 90 is the same as the form of the photoacoustic wave signal sequence 84.

The synchronization deviation is calculated based on the photoacoustic wave signal sequence 84, and at least one of the optical pulse period or the reception period is changed based on the synchronization deviation. As a result, the photoacoustic wave signal sequence 84 is parallel-moved in the depth direction in the coordinate space shown in FIG. 4 (see reference numeral 86). Reference numeral 88 indicates a photoacoustic wave signal sequence observed in a case in which the synchronization deviation is eliminated.

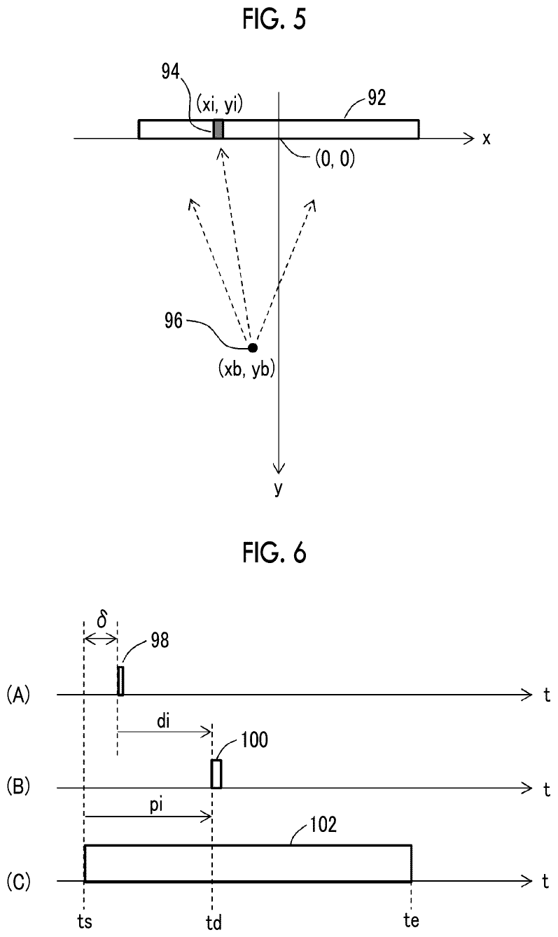

Hereinafter, a calculation method of the synchronization deviation will be described. FIG. 5 shows a spatial relationship between the transducer array 92 and the sound source 96. The x direction is the transducer array direction, and the y direction is the depth direction. The center of the transducer array 92 is the origin (0, 0), and the position of the i-th transducer 94 is represented by (xi, yi). However, yi=0. The position of the sound source 96 is represented by (xb, yb). The propagation time di of the photoacoustic wave from the sound source 96 to the i-th transducer is calculated by the following Expression (1). c in Expression (1) is a sound velocity of the ultrasound wave in the medium.

$${d}_{\text{?}} = \frac{\sqrt{{( {x}_{\text{?}} - {x}_{\text{?}} )}^{2} + {y}_{\text{?}}^{2}}}{c} \qquad ( 1 )$$

$$\text{?} \text{indicates text missing or illegible when filed}$$

The apparent propagation time pi is a time obtained by adding the synchronization deviation δ and the propagation time di. That is, the apparent propagation time pi is represented by the following Expression (2).

$${p}_{\text{?}} = δ + \frac{\sqrt{{( {x}_{\text{?}} - {x}_{\text{?}} )}^{2} + {y}_{b}^{2}}}{c} \qquad ( 2 )$$

$$\text{?} \text{indicates text missing or illegible when filed}$$

A relationship between the apparent propagation time pi, the synchronization deviation 8, and the propagation time (real propagation time) di, which are represented by Expression (2), is shown in FIG. 6. The reception period 102 is a period from the start timing ts to the end timing te. A time between the start timing ts and the generation timing of the optical pulse 98 is the synchronization deviation 8. A propagation time from the generation timing of the optical pulse 98 (generation timing of the photoacoustic wave) to reception of the photoacoustic wave by the i-th transducer is di. Reference numeral 100 indicates the photoacoustic wave signal generated by the reception of the photoacoustic wave.

In a case in which the photoacoustic wave reaches the n pieces of transducers, the n pieces of detection timings td corresponding to the n pieces of transducers are specified. The n pieces of apparent propagation times pi are specified based on the n pieces of detection timings td. The n pieces of data pairs (xi, pi) are defined by the positions xi of the n pieces of transducers and the n pieces of apparent propagation times pi (where, i=1, . . . , n).

The unknown parameters δ, xb, and yb can be specified by substituting the n pieces of data pairs (xi, pi) into Expression (2) which is a mathematical model. In this case, a solution search method such as a least squares method is used. In this method, in addition to the synchronization deviation 8, the coordinates (xb, yb) of the sound source are also specified.

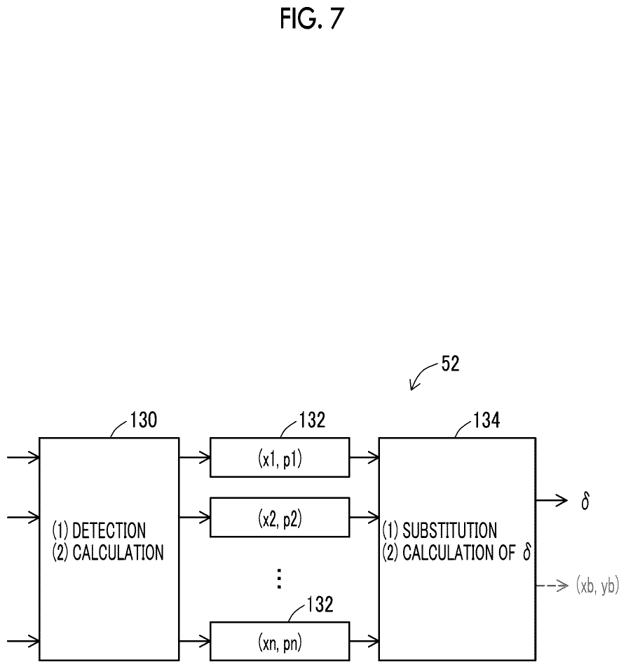

The method described above is schematically shown in FIG. 7. In the analyzer 52, in the block 130, the n pieces of photoacoustic wave signals included in the n pieces of reception signals before phase addition are detected, and the n pieces of apparent propagation times pi from the reception period start timing to the n pieces of detection timing are calculated. The n pieces of data pairs (xi, pi) 132 are defined by the coordinates xi of the n pieces of vibration elements in the x direction and the n pieces of apparent propagation times pi. In the block 134, the calculation of the synchronization deviation δ, which is the unknown parameter, is performed by substituting the n pieces of data pairs (xi, pi) 122 into Expression (2). The coordinates (xb, yb) of the sound source are also calculated secondarily. Only a plurality of data pairs (xi, pi) 122 satisfying certain conditions may be substituted into Expression (2) among the n pieces of data pairs (xi, pi) 122.

The analyzer shown in FIG. 1 calculates the synchronization deviation δ by analyzing the plurality of reception signals. The synchronization controller shown in FIG. 1 changes one of the optical pulse period or the reception period based on the synchronization deviation δ. In a case in which the optical pulse period is changed, the reception period can be maintained, so that there is an advantage that it is not necessary to change the transmission and reception sequence in the ultrasound diagnostic apparatus. In a case in which the reception period is changed, communication between the information processing apparatus (or the ultrasound diagnostic apparatus) and the optical pulse generation apparatus is not required, so that the configuration of the ultrasonic imaging system can be simplified.

It should be noted that, in a case of focusing on |xi−xb| in Expression (2), in many cases, xi−xb|<<yb is established. Therefore, the following Expression (3) is established for Expression (2).

$${p}_{\text{?}} = δ + \frac{{y}_{b}}{c} \sqrt{1 + {( \frac{{x}_{\text{?}} - {x}_{b}}{{y}_{b}} )}^{2}} ≃ δ + \frac{{y}_{b}}{c} + \frac{1}{2 {y}_{b} c} {( {x}_{\text{?}} - {x}_{b} )}^{2} \qquad ( 3 )$$

$$\text{?} \text{indicates text missing or illegible when filed}$$

Expression (3) indicates that the plurality of photoacoustic wave signals draw a parabola in the xy coordinate system. The synchronization deviation δ may be specified by using the above-described Expression (3). The photoacoustic image may be generated by using the coordinates (xb, yb) of the specified sound source, or the operation of the ultrasonic imaging system may be controlled based on the coordinates (xb, yb) of the specified sound source.

It should be noted that, in the synchronization control, instead of directly referring to the synchronization deviation calculated at each point in time, the smoothed synchronization deviation may be referred to. In that case, for example, the smoothed synchronization deviation may be calculated according to Expression (4).

$${PRT}_{j \to \text{?} + 1} = PRT + \frac{1}{N} {\sum }_{\text{?}}^{N - 1} {δ}_{\text{?}} \qquad ( 4 )$$

$$\text{?} \text{indicates text missing or illegible when filed}$$

In Expression (4), δⱼ represents the j-th synchronization deviation. PRTⱼ₊₁ indicates a (j+1)-th optical pulse period calculated based on the j-th optical pulse period. In Expression (4), the N pieces of synchronization deviation amounts are averaged, and an average value of the synchronization deviation amounts is added to the j-th optical pulse period. The optical pulse period smoothed based on another calculation expression may be calculated.

A constant coefficient (for example, 0.8) may be multiplied by the calculated synchronization deviation or the smoothed synchronization deviation, and the optical pulse period or the reception period may be corrected based on the synchronization deviation obtained by the multiplication.

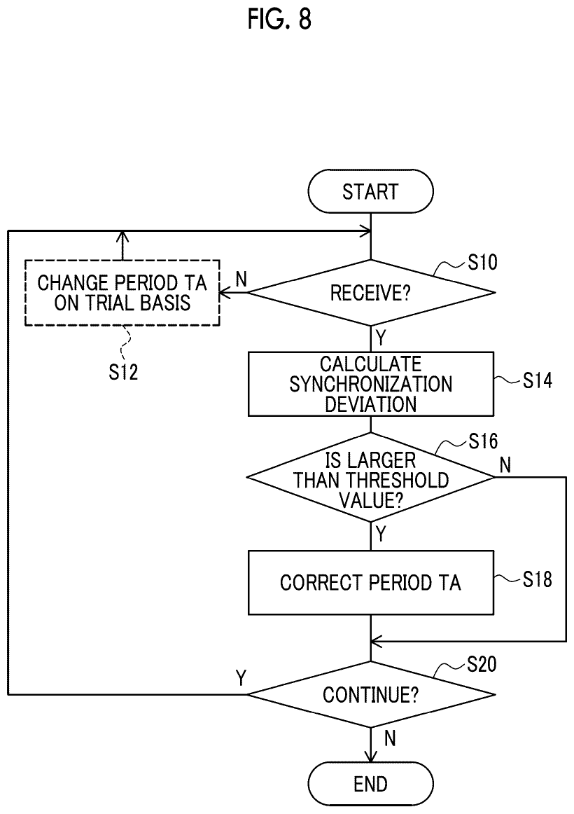

FIG. 8 shows a first example of the synchronization control method as a flowchart. In S10, it is determined whether or not the reception signal sequence before phase addition includes the photoacoustic wave signal sequence. That is, it is determined whether or not the photoacoustic wave signal is received. For example, the presence or absence of the photoacoustic wave signal sequence is determined based on a result of the threshold value processing for each reception signal.

As a case in which the photoacoustic wave signal is not received, a first case and a second case are considered. The first case is a case in which the sound source is not present in an observation region of the ultrasound probe. The second case is a case in which the sound source is present in the observation region of the ultrasound probe, but the reception timing of the photoacoustic wave deviates from the reception period due to the synchronization deviation.

S12 is a step of dealing with the second case. In S12, for example, a constant time is added to the optical pulse period TA, and thus a new optical pulse period is set. Then, S10 is executed again. For example, S12 may be executed by receiving a predetermined input from the user in a case in which the sound source approaches the ultrasound probe. In that case, in a case in which the sound source does not approach the ultrasound probe, it is possible to avoid unnecessary change of the optical pulse period TA. As will be described below, in a case in which the sound source is located directly below the ultrasound probe to establish the synchronization, S12 may be allowed to execute from the beginning.

In S14, the synchronization deviation is calculated based on the photoacoustic wave signal sequence included in the reception signal sequence before phase addition. In S16, it is determined whether or not the synchronization deviation is larger than a predetermined threshold value. In a case in which the synchronization deviation is equal to or less than the predetermined threshold value, the optical pulse period TA is maintained. In a case in which the synchronization deviation is larger than the predetermined threshold value, the optical pulse period TA is changed in S18. In S20, it is determined whether or not to continue the main process. In a case in which it is determined to continue the main process, each step after S10 is executed again.

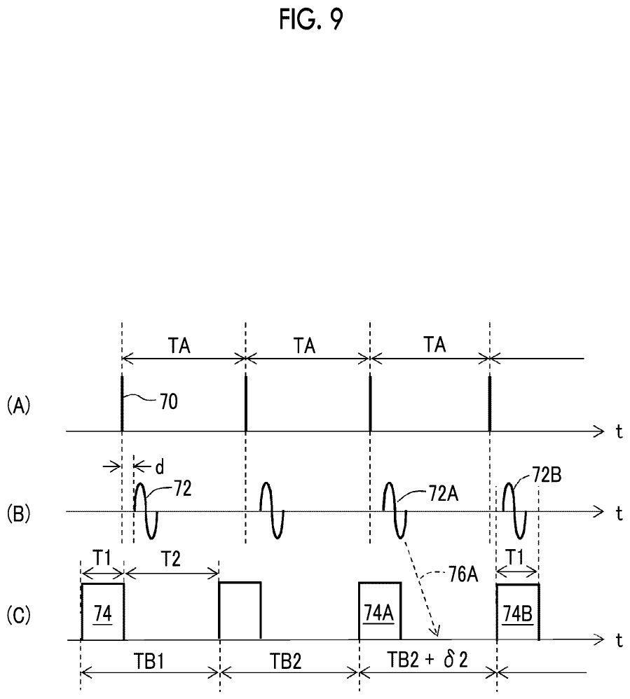

FIG. 9 shows a second example of the synchronization control method as a timing chart. In FIG. 9, the same elements as the elements shown in FIG. 2 are designated by the same reference numerals, and the description thereof will be omitted.

In FIGS. 9, TB1, TB2, and TB2+δ2 indicate the reception periods, respectively. The optical pulse period TA is fixed. In a case in which the photoacoustic wave signal sequence 72 is not detected in the reception period 74, the reception period is changed on a trial basis. Specifically, the next reception period TB2 is set by adding a certain period to the reception period TB1.

In the reception period 74A, the photoacoustic wave signal sequence 72A is observed. The synchronization deviation δ2 is calculated by analyzing the photoacoustic wave signal sequence 72A (see reference numeral 76A), and the synchronization deviation δ2 is added to the current reception period TB2 to set a new reception period TB2+δ2. As a result, synchronization is established between the optical pulse period and the reception period. Specifically, the photoacoustic wave signal sequence 72B is observed in the reception period 74B.

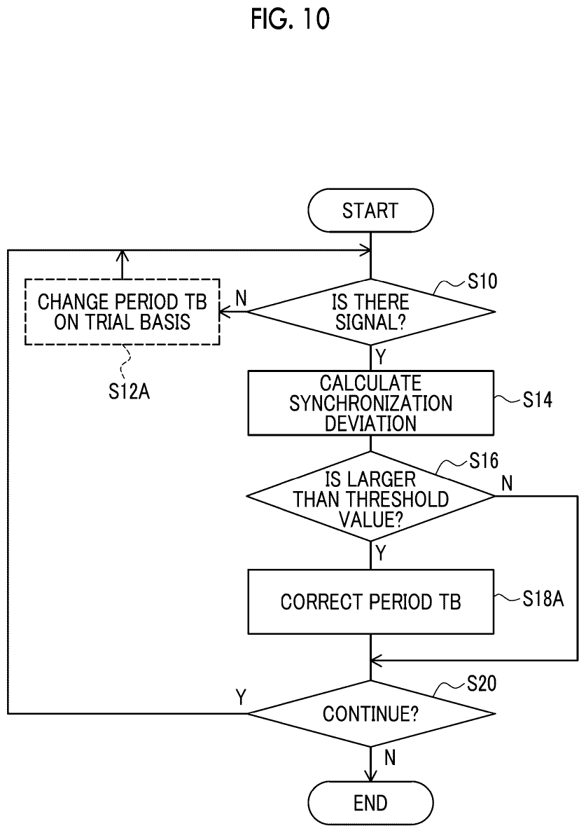

FIG. 10 shows a second example of the synchronization control method as a flowchart. In FIG. 10, the same steps as the steps shown in FIG. 8 are designated by the same step reference numerals, and the description thereof will be omitted. In this second example, the reception period TB is changed on a trial basis in S12A. In addition, in S18A, the reception period TB is corrected based on the synchronization deviation.

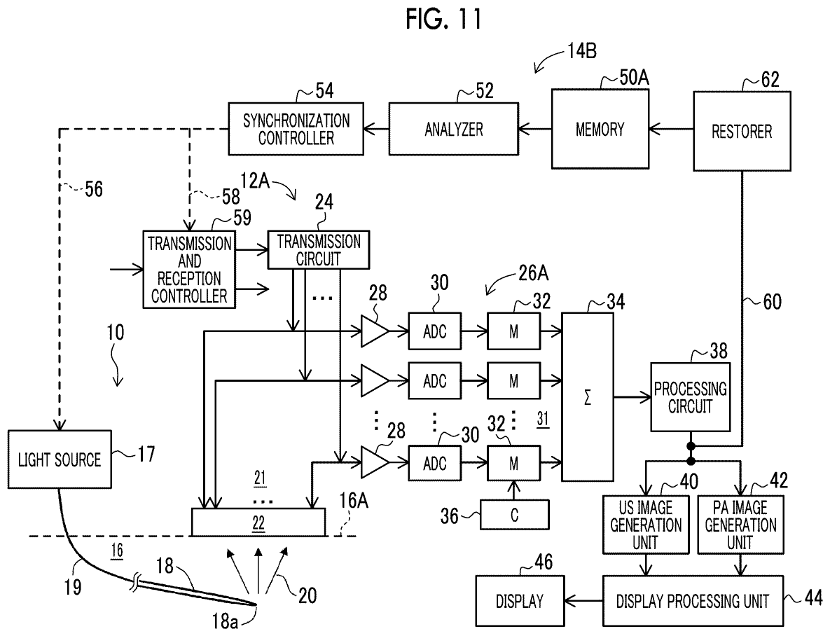

FIG. 11 shows an ultrasonic imaging system according to a second embodiment. In FIG. 11, the same elements as the elements shown in FIG. 1 are designated by the same reference numerals, and the description thereof will be omitted.

In the second embodiment, reception information 60 output from the processing circuit 38 is transferred to the information processing apparatus 14B. The reception information 60 is a plurality of pieces of reception beam data that have undergone a certain process. The reception information output from the reception circuit 26A may be transferred to the information processing apparatus 14B. The transfer data amount can be significantly reduced by transferring the plurality of pieces of reception beam data after the envelope detection.

The information processing apparatus 14B includes a restorer 62, a memory 50A, an analyzer 52, and a synchronization controller 54. The restorer 62 applies a calculation that is the inverse of phase addition to the reception information 60. The calculation that is the inverse of phase addition is also referred to as an inverse Fourier transform or inverse phase addition. The restorer 62 generates a pseudo-reception signal sequence corresponding to the reception signal sequence before phase addition. The pseudo-reception signal sequence is temporally stored in the memory 50A.

In a case in which the pseudo-reception signal sequence is mapped onto the xy coordinate space, the same photoacoustic wave signal sequence as the photoacoustic wave signal sequence shown in FIG. 4 is generated. The analyzer 52 calculates the synchronization deviation based on the photoacoustic wave signal sequence included in the pseudo-reception signal sequence by using the above-described method. The synchronization controller 54 changes the optical pulse period or the reception period based on the synchronization deviation.

According to the second embodiment, there is an advantage that the transfer information can be easily extracted from the ultrasound diagnostic apparatus. The reception information or the PA image after the coordinate transformation may be transferred to the information processing apparatus. Alternatively, information output from the display processing unit 44 may be transferred to the information processing apparatus 14B. In those cases, the pseudo-reception signal sequence is generated based on the transferred information. All or a part of the configuration from the restorer 62 to the synchronization controller 54 may be provided in the ultrasound diagnostic apparatus 12A.

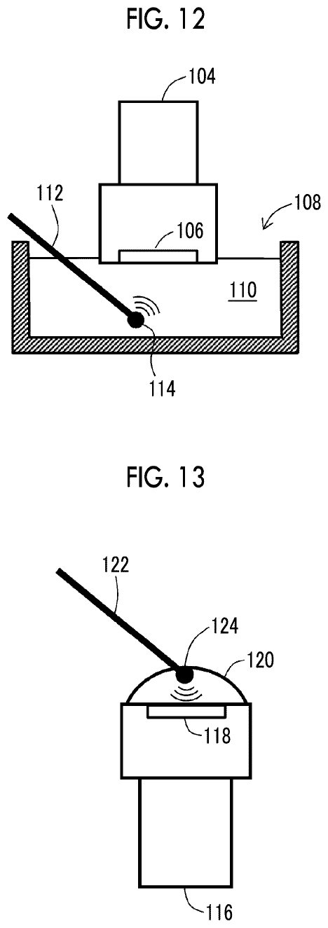

Control of establishing the synchronization may be executed using an acoustic propagation medium other than the living body. Regarding this, FIG. 12 shows a first modification example, and FIG. 13 shows a second modification example.

In the first modification example shown in FIG. 12, the inside of the water tank 108 is a container that stores water 110 as the acoustic propagation medium. A transmission and reception surface of the ultrasound probe 104 is in contact with a surface of the water 110, or a distal end part of the ultrasound probe 104 is inserted into the water 110. In that state, the insertion member 112 is inserted into the water 110, and the sound source 114 is positioned directly below the ultrasound probe 104, specifically, directly below the transducer array in the ultrasound probe 104. In that state, the photoacoustic wave is generated. The photoacoustic wave is detected by the plurality of transducers constituting the transducer array 106. The synchronization deviation is calculated based on the reception signal sequence thus obtained, and the optical pulse period or the reception period is corrected based on the calculated synchronization deviation.

In the second modification example shown in FIG. 13, the ultrasound probe 116 is held such that the transmission and reception surface of the ultrasound probe 116 faces upward. A large amount of the acoustic gels 120 are provided on the transmission and reception surface. For example, the acoustic gel 120 is introduced such that a bulge of the acoustic gel 120 occurs on the transmission and reception surface. Thereafter, the optical absorption element of the insertion member 122, that is, the sound source 124 is inserted into the acoustic gel 120. In that state, the photoacoustic wave is generated. The photoacoustic wave is detected by the plurality of transducers constituting the transducer array 118. The synchronization deviation is calculated based on the reception signal sequence thus obtained, and the optical pulse period or the reception period is corrected based on the calculated synchronization deviation.

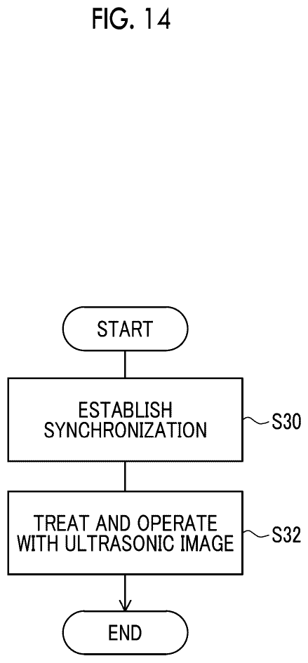

FIG. 14 shows an operation of the ultrasonic imaging system. S30 is a preparation step. In S30, synchronization between the optical pulse period and the reception period is established by using the above-described method. S32 is the main step. In S32, the living body is treated or the living body is examined by using the insertion member. In S32, the optical pulse period and the reception period may be fixed, or the above-described method may be continuously applied to maintain the synchronization establishment state.

As described above, with the ultrasonic imaging system according to the embodiment, it is possible to correctly synchronize an optical pulse period and a reception period while avoiding or reducing complication of a system configuration.

What is claimed is:

1. An ultrasonic imaging system comprising:
a light source configured to generate an optical pulse;
an insertion member that is inserted into a living body and that includes an optical absorption element which converts the optical pulse into a photoacoustic wave;
a probe including a plurality of transducers that receive the photoacoustic wave;
a receiver configured to apply phase addition to a reception signal sequence consisting of a plurality of reception signals that are output in parallel from the plurality of transducers;
a generator configured to generate a photoacoustic image representing a position of the optical absorption element in the living body based on reception information output from the receiver;
an analyzer configured to analyze the reception signal sequence or a pseudo-reception signal sequence corresponding to the reception signal sequence to calculate a synchronization deviation between an optical pulse period in the light source and a reception period in the probe; and
a controller configured to change at least one of the optical pulse period or the reception period based on the synchronization deviation.

2. The ultrasonic imaging system according to claim 1,
wherein the analyzer is configured to
detect a plurality of photoacoustic wave signals included in the reception signal sequence or the pseudo-reception signal sequence, and
calculate the synchronization deviation based on a plurality of detection timings of the plurality of photoacoustic wave signals.

3. The ultrasonic imaging system according to claim 2,
wherein the analyzer configured to
calculate a plurality of apparent propagation times from a reception period start timing to the plurality of detection timings, and
calculate the synchronization deviation based on the plurality of apparent propagation times.

4. The ultrasonic imaging system according to claim 3,
wherein the analyzer is configured to calculate the synchronization deviation by providing the plurality of apparent propagation times to a mathematical model including a synchronization deviation parameter as an unknown parameter.

5. The ultrasonic imaging system according to claim 1,
wherein the analyzer is configured to analyze the reception signal sequence extracted from the receiver.

6. The ultrasonic imaging system according to claim 1, further comprising:
a restorer configured to generate the pseudo-reception signal sequence corresponding to the reception signal sequence based on the reception information,
wherein the analyzer is configured to analyze the pseudo-reception signal sequence.

7. The ultrasonic imaging system according to claim 1,
wherein the controller is configured to, in a case where a photoacoustic wave signal sequence is not included in the reception signal sequence or the pseudo-reception signal sequence, change on a trial basis at least one of the optical pulse period or the reception period until the photoacoustic wave signal sequence is included in the reception signal sequence or the pseudo-reception signal sequence.

8. The ultrasonic imaging system according to claim 1,
wherein the controller is configured to change at least one of the optical pulse period or the reception period based on the synchronization deviation in a preparation step before a main step of performing treatment or examination on the living body.

9. A synchronization control method comprising:
a step of, in a state in which an insertion member including an optical absorption element that converts an optical pulse from a light source into a photoacoustic wave is inserted into an acoustic propagation medium and a probe is in contact with the acoustic propagation medium, receiving the photoacoustic wave from the optical absorption element via a plurality of transducers in the probe;
a step of analyzing a reception signal sequence consisting of a plurality of reception signals output in parallel from the plurality of transducers or a pseudo-reception signal sequence corresponding to the reception signal sequence to calculate a synchronization deviation between an optical pulse period in the light source and a reception period in the probe; and
a step of changing at least one of the optical pulse period or the reception period based on the synchronization deviation.
